(19) United States
(12) Patent Application Publication
(10) Pub. No.: US 2025/0185886 A1
(43) Pub. Date: Jun. 12, 2025

(54) SYSTEMS, DEVICES, AND METHODS FOR DETECTION OF ENDOSCOPY LOOPING AND BOWING

(71) Applicant: Boston Scientific Scimed, Inc., Maple Grove, MN (US)

(72) Inventors: Kian S. LIM, Shrewsbury, MA (US); Longquan CHEN, Lexington, MA (US)

(73) Assignee: Boston Scientific Scimed, Inc., Maple Grove, MN (US)

(21) Appl. No.: 18/968,693

(22) Filed: Dec. 4, 2024

Related U.S. Application Data
(60) Provisional application No. 63/606,801, filed on Dec. 6, 2023.

(51) Int. Cl. A61B 1/00; A61B 5/06; A61B 34/20; A61B 90/00; A61M 25/01

(52) U.S. Cl. CPC A61B 1/00055; A61B 5/067; A61B 34/20; A61B 2034/2055; A61B 2090/3937; A61M 2025/0166

(57) ABSTRACT

Disclosed is a system for detecting bowing or looping events during a medical procedure, the system comprising: a medical device including: an insertion portion having a proximal portion and a distal end. A distal sensor may be disposed at the distal end of the insertion portion. The distal sensor is configured to measure an acceleration or a velocity of the distal end of the insertion portion or a distance traveled by the distal end of the insertion portion. A proximal sensor is configured to measure an acceleration or a velocity of the proximal portion of the insertion portion or a distance traveled by the proximal portion of the insertion portion.

### CROSS-REFERENCE TO RELATED APPLICATIONS

This application claims the benefit of priority to U.S. Provisional Application No. 63/606,801, filed on Dec. 6, 2023, which is incorporated by reference herein in its entirety.

### TECHNICAL FIELD

The disclosure relates generally to systems, devices, and methods for detection of looping or bowing of a medical device. More specifically, aspects of the disclosure pertain to devices, systems, and/or methods for detection of looping and/or bowing to generate and display a graphical user interface (GUI) in order to facilitate a medical procedure.

### BACKGROUND

In endoscopic procedures, a medical device (e.g., an endoscope) is inserted into a patient's body to examine and/or perform medical procedures in the interior of a hollow organ or a body lumen of the patient's body. In some endoscopic procedures, particularly those involving gastrointestinal endoscopy, bowing or looping of an endoscope may occur, requiring corrective action by a user before the procedure may continue. Bowing and looping are significant causes of patient discomfort and of prolonged or failed procedures. Early detection of possible bowing or looping is important to minimizing the disruption to the procedure they cause. Thus, there exists a need for systems and methods for detection of looping or bowing of a medical device, such as an endoscope.

### SUMMARY

This disclosure includes medical systems and methods for determining looping or bowing of an endoscope.

In an example, a system for detecting bowing or looping events during a medical procedure may include a medical device. The medical device may include: an insertion portion having a proximal portion and a distal end; and a distal sensor disposed at the distal end of the insertion portion. The distal sensor may be configured to measure an acceleration or a velocity of the distal end of the insertion portion or a distance traveled by the distal end of the insertion portion. The system may also include a proximal sensor configured to measure an acceleration or a velocity of the proximal portion of the insertion portion or a distance traveled by the proximal portion of the insertion portion.

Any of the aspects disclosed herein may include any of the following features, alone or in any combination. The distal sensor may be an inertial measurement unit (IMU). The proximal sensor may include a fiber optic sensor or a camera. The insertion portion may include a plurality of stripes. A first set of the plurality of stripes may have a first property, and a second set of the plurality of stripes may have a second property different from the first property. The proximal sensor may be configured to sense a passing of the plurality of stripes to determine the distance traveled by the proximal portion of the insertion portion. The proximal sensor may be configured to sense a passing of the plurality of stripes to determine the velocity of the distal end. The proximal sensor may be attached to a patient's skin. The system may include a controller configured to compare one or more of the acceleration, the velocity, or the distance measured by the distal sensor to the acceleration, the velocity, or the distance measured by the proximal sensor. The comparison may include determining one or more of (a) an acceleration difference between the acceleration measured by the proximal sensor and the acceleration measured by the distal sensor, (b) a velocity difference between the velocity measured by the proximal sensor the velocity measured by the distal sensor, or (c) a distance difference between the distance measured by the proximal sensor and the distance measured by the distal sensor. The controller may be configured to compare one or more of the acceleration difference, the velocity difference, or the distance difference to a respective threshold. The controller may be configured to determine that looping or bowing of insertion portion is occurring upon determining that one of more of the acceleration difference, the velocity difference, or the distance difference is greater than the respective threshold. The controller may be configured to generate an alert upon determining that one of more of the acceleration difference, the velocity difference, or the distance difference is greater than the respective threshold. The alert may be auditory, visual, or tactile. The controller may provide instructions to automatically adjust a scope upon determining that one of more of the acceleration difference, the velocity difference, or the distance difference is greater than a respective threshold. The controller may be configured to determine that looping or bowing of the insertion portion is occurring upon determining that the difference between the proximal distance the distal distance is greater than a predetermined threshold for a predetermined interval of time. The insertion portion may be configured to be inserted into a patient's body and the proximal sensor may be configured to remain outside of the patient's body.

In another aspect, the system may include: a medical device including an insertion portion having a proximal portion and a distal portion. The distal portion may include a first sensor configured to measure a first parameter of the distal portion; and a second sensor configured to measure a second parameter of the proximal portion. The system may also include a controller configured to compare the second parameter and the first parameter in order to determine whether looping or bowing of the insertion portion has occurred or is occurring.

Any of the aspects disclosed herein may include any of the following features, alone or in any combination. The first parameter may be distance, velocity or acceleration and the second parameter may be distance, velocity, or acceleration.

In yet another aspect, a medical method may include receiving a proximal measurement from a proximal sensor. The proximal measurement may indicate a velocity or acceleration of a proximal portion of an insertion portion of a medical device or a distance traveled by the proximal portion of the insertion portion of the medical device. The method may also include receiving a distal measurement from a distal sensor. The distal measurement may indicate a velocity or acceleration of a distal portion of an insertion portion of a medical device or a distance traveled by the distal portion of the insertion portion of the medical device. The method may further include: comparing the proximal measurement to the distal measurement; based on the comparison between the proximal measurement and the distal measurement, determining that a bowing event or a looping event has occurred or is occurring; and providing an alert that the bowing or looping event has occurred or is occurring.

Any of the aspects disclosed herein may include any of the following features, alone or in any combination. The comparing may include determining a difference between the proximal measurement and the distal measurement. The method may include comparing the difference to a predetermined threshold.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an aspect of an exemplary environment where detection of looping and/or bowing of a medical device may be implemented to facilitate a medical procedure.

FIG. 2 depicts an exemplary medical device used in the exemplary environment of FIG. 1.

FIG. 3A depicts an exemplary proximal sensor assembly for use with the environment of FIG. 1.

FIG. 3B depicts an alternative proximal sensor assembly for use with the environment of FIG. 1.

FIG. 4 depicts an exemplary process for detection of looping and/or bowing of an endoscope.

FIG. 5 depicts an exemplary graphical user interface (GUI).

FIG. 6 depicts an example of a computing device.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator/toward a treatment site, and the term “proximal” refers to a direction toward an operator. The term “exemplary” is used in the sense of “example,” rather than “ideal.” The term “approximately,” or like terms (e.g., “substantially”), includes values +/−10% of a stated value.

As briefly described above, during an exemplary endoscopic procedure, particularly a gastrointestinal endoscopic procedure, situations commonly referred to as “bowing” or “looping” may arise. Bowing or looping may cause discomfort or pain to a patient undergoing the procedure, may prolong the procedure by adding significant time required to address the situation, or may ultimately jeopardize a successful procedure if the situation cannot be adequately addressed. These situations are causes of patient pain during gastrointestinal endoscopic procedures, and are also leading causes of prolonged and failed procedures.

In endoscopy, a user inserts an endoscope or other medical device into a patient's body lumen, such as a gastrointestinal tract, and advances the endoscope through the body lumen to either examine a portion of the body lumen or to arrive at a target position in the body lumen to further perform a procedure. In a typical endoscopy procedure, as the user advances the endoscope from a proximal (closer to the user) position outside the patient's body, a distal tip of the endoscope within the patient's body advances within the target body lumen (e.g., the gastrointestinal tract) at a speed and distance equal to the speed and distance that the proximal portion of the endoscope has advanced.

However, in some situations, the distal tip of the endoscope fails to successfully advance. One such situation arises where a portion of the endoscope, rather than advancing within the body lumen, pushes on a side surface of the body lumen, distending that portion of the body lumen and preventing the distal tip from advancing further through the body lumen. Instead, the distal tip may remain stationary relative to a position within the body lumen, while the endoscope loops or bows within the distending body lumen. In some examples, the distal tip may even retract relative to the body lumen because of the distending of the body lumen. This situation is what is referred to as “bowing” or “looping.” “Bowing” and “looping” generally describe a respective shape of the body lumen (bowed or looped) and are used in a generally interchangeable way in this specification.

It may be possible to identify looping or bowing if a user recognizes that an image provided by an imaging device, typically a camera, on the distal tip of the endoscope does not appear to be advancing at the same rate as the proximal portion of the endoscope outside the patient's body. Early identification of potential looping or bowing is useful in preventing escalated situations that require more significant intervention. However, the training and experience required for successful identification of early looping or bowing indicators is significant, and even well-trained and experienced users may have difficulty identifying such situations all of the time. Thus, it is not uncommon for more escalated looping or bowing situations to arise.

More escalated situations require sometimes painful physical interventions, such as external applications of counter-pressure to remedy the distended colon, or torqueing and pulling back of the endoscope, a maneuver known as “Torque right pull back,” which is an advanced maneuver that may require multiple attempts before succeeding in alleviating the looping or bowing effect. In addition to causing potential physical harm and discomfort that a patient may experience after the procedure, these corrective actions also prolong the procedure that is often done under anesthesia.

Thus, there exists a need to better and more quickly identify potential bowing or looping situations during endoscopy procedures.

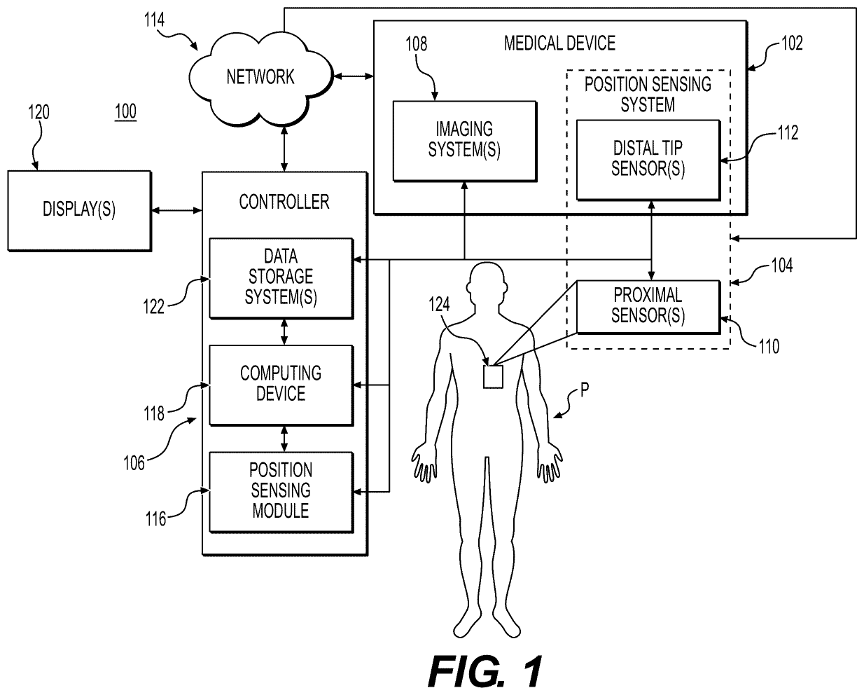

FIG. 1 depicts an exemplary environment 100 where detection of bowing or looping may be implemented to facilitate a medical procedure, such as a gastrointestinal endoscopy. Environment 100 may include a medical device 102, a position sensing system 104, a controller 106, and one or more display(s) 120. In aspects, portions of position sensing system 104 may be integrated into medical device 102 and/or controller 106. Display 102 may be separate from controller 106 (and connected to controller 106 via a wired or wireless connection) or may be integrated into controller 106. Medical device 102 and/or position sensing system 104 may be in wired or wireless communication with controller 106. For example, an umbilicus of medical device 102 may be connected to controller 106. Additionally or alternatively, one or more of medical device 102, position sensing system 104, and/or controller 106 may communicate with each other and one or more other components of environment 100 over a wired or wireless network, such as network 114.

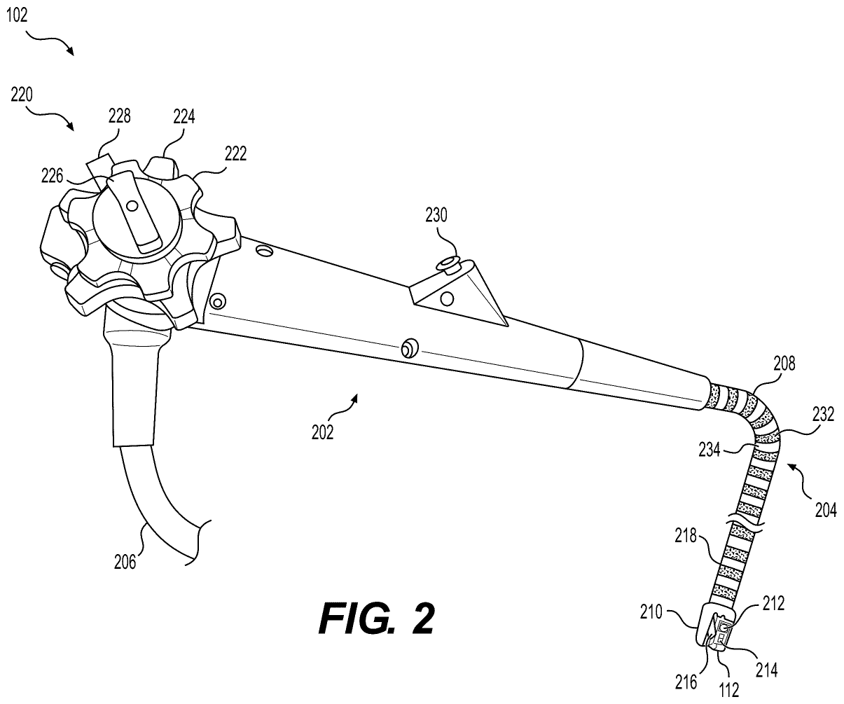

Medical device 102 may be used to perform a medical procedure. Medical device 102 may be an endoscope or other type of scope, and the endoscope may be a specialized type of endoscope utilized for the medical procedure. For example, medical device 102 may be a gastroscope. Although an endoscope or a gastroscope may be referred to herein, it will be appreciated that medical device 102 may be any suitable type of medical device (e.g., duodenoscope, colonoscope, bronchoscope, ureteroscopes, cystoscope, cholangioscopes, tome, catheter, etc.). Medical device 102 may have any of the features of any of the exemplary medical devices listed above. In some examples, medical device 102 may include one or more position sensing components of position sensing system 104, such as distal tip sensor(s) 132, described in more detail below. Medical device 102 may also include imaging system(s) 108 for capturing images of anatomy of patient P, described in more detail with reference to FIG. 2.

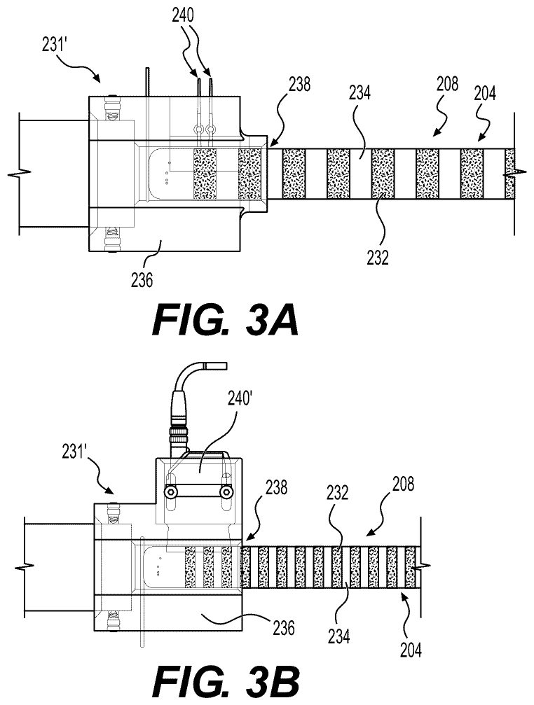

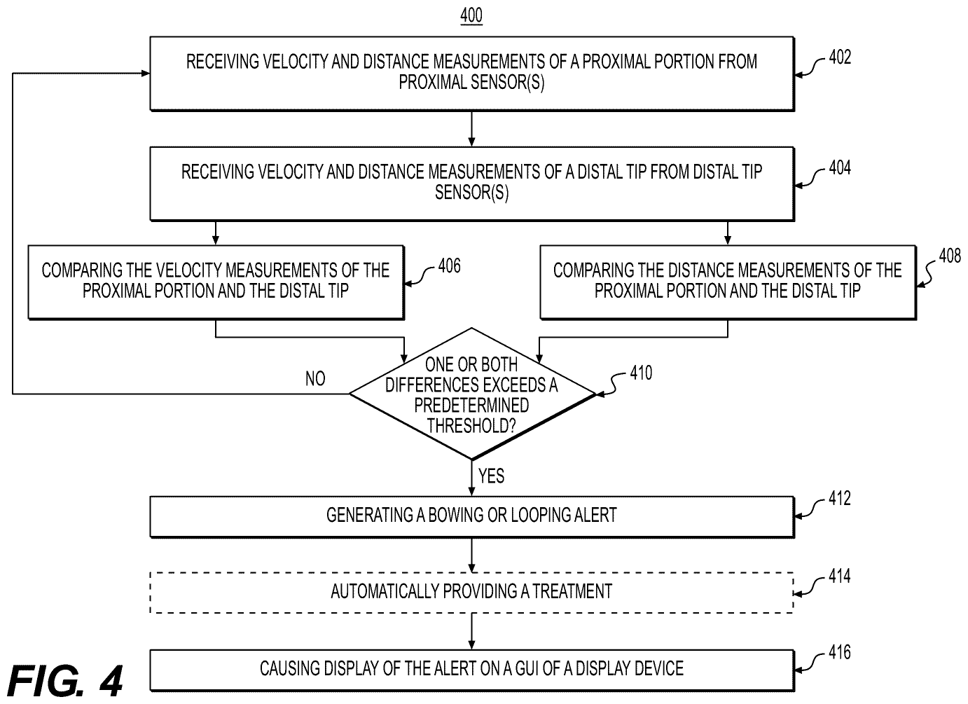

Position sensing system 104 may be a tracking system for determining a position and/or orientation of medical device 102 within the body of patient P. Position sensing system 104 is described in further detail with respect to FIGS. 2 and 3 below. Position sensing system 104 may include one or more proximal sensor(s) 110 (e.g., proximal sensor(s)) and one or more distal tip sensor(s) 112 configured to measure and track the distance, velocity, and/or acceleration of respective proximal and distal portions of medical device 102 as it is inserted into a patient P. Proximal sensor(s) 110 may be positioned at a position outside the body of patient P (e.g., as a patch 124 on a skin of patient P), to measure and track the distance, velocity, and/or acceleration of a portion of the medical device outside the body of patient P, at a proximal position relative to the distal tip of the medical device 102. Distal tip sensor(s) 112 may be positioned at a distal tip of the medical device and configured for measuring a distal velocity, distance traveled, and/or acceleration of the medical device 102. By comparing the values generated by the proximal sensor(s) 110 proximally and the distal tip sensor(s) 112 distally, possible looping or bowing may be detected based on the different measurements, as described in further detail below with respect to FIG. 4.

With further reference to FIG. 1, controller 106 may be a stand-alone computing device configured to communicate with one or more of the other components of environment 100. For example, medical device 102 and/or elements of position sensing system 104 may be connected to controller 106 by wireless or wired connections. In other examples, Controller 106 may communicate across network 114 with one or more of medical device 102, position sensing system 104, or display 120, to exchange information, including to receive image data from imaging system 108 and spatial data (e.g., position and/or orientation information) from position sensing system 104. Controller 106 may be a computer system, such as, for example, a specialized hardware device, desktop computer, a laptop computer, a tablet, a smart cellular phone, a smart watch or other electronic wearable, etc. In alternatives, controller 106 may be a distributed system, and elements of controller 106 may be disposed in separate physical structures/separate locations.

Controller 106 (e.g., computing device 118 of controller 106) may be communicatively coupled to medical device 102 to transmit and receive signals from medical device 102. For example, controller 106 may transmit signals to cause one or more illumination devices (see FIG. 2) of medical device 102 to illuminate an area of interest within a body lumen of a patient P. Additionally, controller 106 may receive image signals from one or more imaging devices of imaging system 108 of medical device 102.

Controller 106 may include a position sensing module 116, a computing device 118, and a data storage system 122. Although position sensing module 116, computing device 118, and data storage system 122 are described as separate components, they may be combined with one another. For example, position sensing module 116 may be an element of computing device 118. Furthermore, controller 106 (e.g., computing device 118) may include other elements not separately described. Elements of position sensing module 116, computing device 118, and data storage system 122 may be on one or more circuit boards of controller 106.

Controller 106 (e.g., computing device 118 of controller 106) may have one or more applications (e.g., software programs) locally installed for performing image processing that may be executed to process the image signals to generate an image (e.g., a live image) for display on one or more of display(s) 120 communicatively coupled to controller 106. For example, as medical device 102 is inserted into and navigated toward a target site through a body lumen of patient P, a plurality of image signals may be received from an imaging device of imaging system 108 and processed by controller 106 (e.g., by computing device 118 of controller 106) to generate and cause display of a plurality of corresponding images.

In some examples, controller 106 may have one or more additional applications (e.g., software programs, plug-ins, etc.) installed locally to perform one or more operations associated with detection of looping or bowing (e.g., one or more operations described in FIG. 4). For example, such applications may be installed locally on position sensing module 116 or on computing device 118. For example, measurements from proximal sensor(s) 110 and distal tip sensor(s) 112 may be processed to produce display elements and/or alerts. In other examples, the measurements may be transmitted to another system and/or computing device within environment 100 or outside of environment 100 for processing, analysis, storage, display, etc.

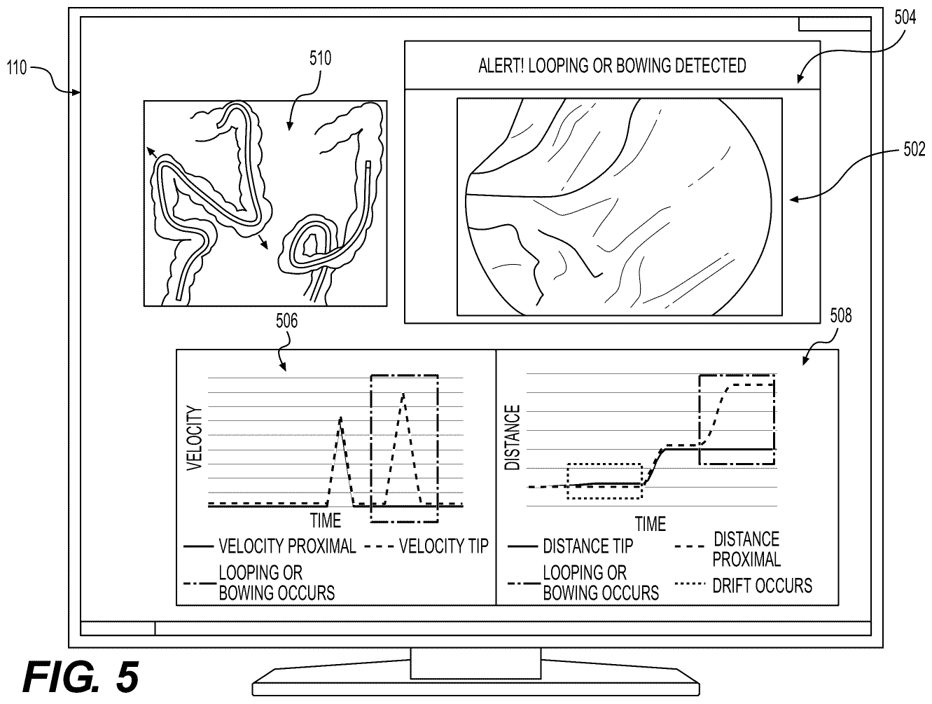

One or more components of controller 106 (e.g., of computing device 118 of controller 106), such as one of the applications, may generate, or may cause to be generated, one or more GUIs based on instructions/information stored in memory, e.g., on data storage system(s) 122, instructions or information received from the other components in environment 100, and/or the like. One or more components of controller 106 may also cause the GUIs as described in FIG. 5 to be displayed via one of display(s) 120. The GUIs may include images, text, input text boxes, selection controls, and/or the like. Display 120 may include a touch screen or a display with other input systems (e.g., a mouse, keyboard, etc.) for a user to control the functions of controller 106.

Controller 106 (e.g. computing device 118 of controller 106) may include one or more applications (e.g., software programs) locally installed on, e.g., a memory of computing device for performing image processing that may be executed to generate images. Additionally, one or more components of controller 106, such as one of the applications, may generate, or may cause to be generated, one or more GUIs based on instructions/information stored in the memory, instructions/information received from the other components in environment 100, and/or the like. The one or more components of controller 106 may cause the GUIs to be displayed via one of display(s) 120. The GUIs may include images, text, input text boxes, selection controls, and/or the like, and may enable operator interaction with the images captured by a respective modality from the various above-described modalities.

For example, controller 106 (e.g., computing device 118 and/or position sensing module 116) may generate and display the information received from position sensing system 104 (including alerts based on the information received) and/or imaging system 108 on display(s) 120, and a user may utilize an application to view or interact with images or alerts produced by position sensing system 104.

Data storage system 122 may include a server system or computer-readable memory, such as a hard drive, flash drive, disk, etc. Data storage system 122 includes and/or interacts with an interface for exchanging data to other systems, e.g., one or more of the other components of environment 100 (e.g., components of controller 106). For example, data storage system 122 may be configured to receive and store information from position sensing system 104 for patient P or images generated by one or more of imaging system(s) 108. As another example, data storage system 122 may be configured to receive and store the plurality of images of the body lumen of patient P from medical device 102.

Display(s) 120 may be communicatively coupled to one or more other components of environment 100 to receive and display data, including image data and position sensing data. For example, display 120 may be connected via wired or wireless connection to controller 106. In some aspects, display 120 may be integrated into controller 106 and may not be a separate structure from controller 106. Display(s) 120 may receive and display the image that was captured by one of imaging system(s) 108 (e.g., as medical device 102 is being inserted into and navigated toward the target site through the body lumen of patient P). Additionally, display(s) 120 may receive and display position data from position sensing system 104 received from proximal sensor(s) 110 and/or distal tip sensor(s) 112.

The one or more applications executed on the one or more components of environment 100 are described herein as local applications that are installed, e.g., on a memory of the respective components such that a network connection (e.g., Internet access) is not required to enable communication with a remote server and the application to function. However, in other embodiments, the applications may be web-based applications that are accessible via a browser executing on the component, where the one or more application may communicate with a remote server (not shown) over network 114. In such examples, one or more operations of the 3D image registration may be performed by processing devices of the remote server.

As mentioned, the one or more components of environment 100 may communicate over network 114. Network 114 may be an electronic network. Network 114 may include one or more wired and/or wireless networks, such as a wide area network (“WAN”), a local area network (“LAN”), personal area network (“PAN”), a cellular network (e.g., a 3G network, a 4G network, a 5G network, etc.), or the like. In one non-limiting, illustrative example, the components of environment 100 may communicate and/or connect to network 114 over universal serial bus (USB) or other similar local, low latency connections or direct wireless protocol.

In some embodiments, network 114 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. Components of environment 100 may be connected via network 114, using one or more standard communication protocols such that the component may transmit and receive communications from each other across network 114, as discussed in more detail below.

Although various components in environment 100 are depicted as separate components in FIG. 1, it should be understood that a component or portion of a component in environment 100 may, in some embodiments, be integrated with or incorporated into one or more other components. In some embodiments, operations or aspects of one or more of the components discussed above may be distributed amongst one or more other components. Any suitable arrangement and/or integration of the various systems and devices of environment 100 may be used.

FIG. 2 depicts an exemplary medical device 102 of FIG. 1. Medical device 102 may include a handle 202 and an insertion portion 204. Medical device 102 may also include an umbilicus 206 for purposes of connecting medical device 102 to sources of, for example, air, water, suction, power, etc., as well as to image processing and/or viewing equipment, such as controller 106.

Insertion portion 204 may include a sheath or shaft 208. Shaft 208 may be striped, including alternating stripes 232 and 234 along a portion or an entirety of shaft 208 of insertion portion 204 for purposes of providing a detectable marker for proximal sensor(s) 110. As will be further described in FIG. 3 below, proximal sensor(s) 110 may include one or more encoders that have sensors that are configured to detect the stripes 232 and 234 of shaft 208 as they pass the sensors. Proximal sensor(s) 110 may determine and transmit, based on these measurements, an acceleration, velocity, and/or distance traveled of/by the shaft. The stripes 232 and 234 may be of alternating color or luminosity. For examples, stripes 232 may have a first color and stripes 234 may have a second color (e.g., stripes 232 may be white while stripes 234 may be black). Alternatively, stripes 232 and 234 may have any other suitable colors or other properties. Stripes 232 and 234 may be evenly spaced apart, e.g., be of substantially equal width or may have varying widths. In examples, stripes 232 may have a first width, and stripes 234 may have a second width. Although two types of stripes 232, 234 are discussed, shaft 208 may alternatively have further types (e.g., colors) of stripes.

A distal portion of shaft 208 that is connected to distal tip 210 may have a steerable section 218. Steerable section 218 may be, for example, an articulation joint. Shaft 208 and steerable section 218 may include a variety of structures (e.g., steering wires and joints), which are known or may become known in the art.

Insertion portion 204 may also include distal tip 210. Distal tip 210 may include one or more components of imaging system(s) 108, such as imaging devices 212 (e.g., one or more cameras) for capturing images, and one or more illumination devices 214 (e.g., one or more light emitting diodes (LEDs) or optical fibers) for providing illumination to facilitate image capture and visualization. Distal tip 210 may also include one or more distal tip sensor(s) 112 for measuring acceleration, velocity, and/or distance traveled of/by distal tip 210. Although a distal tip sensor is shown and described as being on distal tip 210, it will be appreciated that distal tip sensor 112 may alternatively be in a different location, such as on a distal end of shaft 208.

Distal tip 210 may be side-facing. That is, imaging device 212 and illumination devices 214 may face radially outward, perpendicularly, approximately perpendicularly, or otherwise transverse to a longitudinal axis of shaft 208 and distal tip 210. However, this disclosure also encompasses other configurations of distal tips. For example, distal tip may be “forward facing” (i.e., distal-facing).

Imaging device 212, illumination devices 214, and distal tip sensor(s) 112 may be mounted at distal tip 210 by any suitable method, including, but not limited to, wire bonding, surface mount assembly, electro mechanical assembly, and/or plated through-hole technology. Although one imaging device 212 and two illumination devices 214 are depicted in FIG. 2, any suitable number of imaging devices 212 and/or illumination devices 214 may be utilized. Alternatively, imaging device 212 and illumination devices 214 may be combined into a single device. Distal tip sensor(s) 112 may be disposed on an outer surface of distal tip 210 or within an interior of distal tip 210 or within or on a distal portion of shaft 208.

Distal tip sensor 112 may be or may include an accelerometer or inertial measurement unit (IMU) chip. For example, distal tip sensor 112 may be a six degree of freedom IMU chip. Any accelerometer or IMU chip with suitable dimensions (e.g., under approximately 5 mm in height, length, and width) and with at least three degrees of freedom and up to six degrees or more of freedom may be used as distal tip sensor 112. In instances where an accelerometer or IMU chip is used as distal tip sensor 112, controller 106 (e.g., position sensing module 116) may include programming to derive the velocity and distance traveled from the acceleration data obtained by distal tip sensor 112, by performing first integral operations and double integral operations, respectively (discussed in further detail below).

Wires or cables to power imaging device 212, illumination devices 214, and distal tip sensor(s) 133 may extend through shaft 208 and into handle 202. The wires or cable may be connected to controller 106, e.g., via umbilicus 206. For example, controller 106 (e.g., computing device 118) may transmit signals to cause illumination devices 214 to illuminate, may receive image signals from imaging device 212 for processing and subsequent display, and may receive acceleration data from distal tip sensor 112. In some examples, controller 106 (e.g., computing device 118) may also initiate image capture of still images from imaging device 212 by transmitting signals via the wires or cables housed in conduit to cause imaging device 212 to capture an image.

Distal tip 210 may also include an elevator 216 for changing an orientation of a tool inserted in a working channel of medical device 102. Elevator 216 may alternatively be referred to as a swing stand, pivot stand, raising base, or any suitable other term. Elevator 216 may be pivotable via, e.g., an actuation wire or another control element that extends from handle 202, through shaft 208, to elevator 216. Elevator 216 may be pivotable about an axle rotatably retained within distal tip 210.

Distal tip 210 may also include components in addition to or in the alternative to the components described above. For example, distal tip 210 also may include additional or alternative sources of lighting and/or additional or alternative imaging components (e.g., additional cameras). Distal tip 210 may also include additional types of sensors, such as moisture sensors, temperature sensors, pressure sensors, or other types of sensors.

Handle 202 may have one or more actuators/control mechanisms 220. Control mechanisms 220 may provide control over steerable section 218 or may allow for provision of air, water, suction, etc. For example, handle 202 may include control knobs 222, 224 for left, right, up, and/or down control of steerable section 218. For example, one of knobs 222, 224 may provide left/right control of steerable section 218, and the other of knobs 222, 224 may provide up/down control of steerable section 218. Handle 202 may further include one or more locking mechanisms 226 (e.g., knobs or levers) for preventing steering of steerable section 218 in at least one of an up, down, left, or right direction. Handle 202 may include an elevator control lever (not shown). The elevator control lever may raise and/or lower elevator 216, via connection between the lever and an actuating wire (not shown) that extends from lever 228, through shaft 208, to elevator 216. A port 230 may allow passage of a tool (e.g., one of tool(s) 123) through port 230, into a working channel (not shown) of medical device 102, through shaft 208, to distal tip 210. Although not shown, handle 202 may also include one or more valves, buttons, actuators, etc. to control the provision of air, water, suction, etc.

In use, a user may insert at least a portion of shaft 208 of insertion portion 204 into a body lumen of a subject, such as patient P (FIG. 1). Distal tip 210 may be navigated to a target site in the body lumen, using imaging devices 212 for navigation, illumination devices 214 for illumination, and distal tip sensor 112 to help prevent bowing or looping, as discussed below.

FIG. 3A depicts a close-up view of shaft 208 and a proximal sensor assembly 231, which may include sensors having any of the properties of proximal sensor 110. As discussed with reference to FIG. 2 above, shaft 208 may be striped, including alternating stripes 232 and 234 along a portion or an entirety of shaft 208 for purposes of providing a detectable marker for sensors (e.g., proximal sensors 110) of proximal sensor assembly 231. FIG. 3 is shown with a distal end of shaft 208 being inserted into proximal sensor assembly 231. As shaft 208 is advanced distally relative to proximal sensor assembly 231 (e.g., as shaft 208 is inserted into a body lumen of a subject), increasingly proximal portions of shaft 208 may pass through proximal sensor assembly 231.

Proximal sensor assembly 231 shown in FIG. 3 may include a housing 236 that may be mounted to a table or held by an operator, with a lumen or opening 238 for receiving shaft 208 of insertion portion 204 of the medical device 102. Proximal assembly 231 may be positioned, for example, on a table or other flat surface near patient P (e.g., near a natural orifice or incision through which insertion portion 204 will be inserted).

Proximal sensor assembly 231 further includes one or more sensors 240 that are configured to detect stripes 232 and 234 as they pass by proximal sensor(s) 110. Sensors 240 may be used as proximal sensors 110 or may operate together to form with other elements proximal sensor 110. In some examples, sensors 240 may be fiber optic sensors (e.g., two fiber optic sensors), which may measure a distance traveled by the insertion portion 204 by counting stripes 232 and 234 as they pass sensors 240.

Proximal sensor assembly 231 (e.g., sensors 240 and associated hardware or software) may be configured to detect the direction of motion of insertion portion 204. This allows for counting up as insertion portion 204 is inserted into the body lumen of patient P, for example, as insertion portion 204 moves to the left in FIG. 3, and for counting down as insertion portion 204 is retrieved from the body lumen of patient P, e.g., as insertion portion 204 moves to the right in FIG. 3.

Together with a time register that may be onboard proximal sensor assembly 231 or an element of controller 106, sensor(s) 240 may directly measure the velocity of insertion portion 204 by dividing the distance between two stripes 232 and 234 as they pass fiber optic sensors 240 by the time elapsed between measurements. Proximal sensor assembly 231 may determine and transmit, based on these measurements, an acceleration, velocity, and/or distance traveled of shaft 208. Stripes 232 and 234 may be of alternating color or luminosity, e.g., stripes 232 may be white while stripes 234 are black. Stripes 232 and 234 may be evenly spaced apart, e.g., be of substantially equal width, to simplify the measurements.

FIG. 3B shows an alternative proximal sensor assembly 231′ having any of the features of proximal sensor assembly 230 unless otherwise specified. Instead of sensors 240 (e.g., optical sensors), proximal sensor assembly 231′ may include a sensor 240′ (or a plurality of sensors 240′). Sensor 240′ may include a camera that detects the stripes 232 and 234 by identifying the change in color between stripes. Sensor assembly 231′ may function similarly to sensor assembly 231 but by using sensor 240′ to measure acceleration, velocity, and/or distance. For example, sensor 240′ or other components of proximal sensor assembly 231′ may measure a time difference between when a stripe 232 and a stripe 234 is detected by sensor 240′ or may detect passing boundaries between stripe 232 and stripe 234.

In some examples, proximal sensor assemblies 231 or 231′ may be configured as a patch 124 (shown in FIG. 1). For example, such a configuration may be used where insertion portion 204 is inserted through an incision in a patient's skin, instead of via a natural orifice. Patch 124 may be positioned on the patient P where the insertion portion 204 is inserted into the patient P. Alternatively, patch 124 may include a type of sensor that is not an encoder (e.g., a radiofrequency sensor, magnetic sensor, or the like) that is configured to interact with elements of insertion portion 204 (e.g., tags, magnets, other sensors, or the like). Such elements may be positioned at known positions of shaft 208, such that proximal sensors 110 of patch 124 may measure speed, velocity, acceleration, position, etc. of shaft 208. Stripes 232, 234 may include such elements.

The distance measurement may represent the length of insertion portion 204 that has passed by the position of the proximal sensor(s) 110 (a distance between distal tip 210 and proximal sensor 110) in a time t from the beginning of the procedure.

The types of sensors described above are not limiting. Proximal sensor 110 may additionally or alternatively be an IMU or other type of sensor coupled to a proximal portion of shaft 208. In examples, a plurality of sensors 110 may be coupled along a length of shaft 208 at fixed or varying distances.

FIG. 4 depicts an exemplary process 400 for detection of bowing or looping of an endoscope during a medical procedure. Process 400 may be performed by controller 106 and, more specifically, by computing device 118 and/or position sensing module 116. Elements of controller 106 are described as including application(s) configured to perform one or more operations or steps of process 400. Additionally or alternatively, one or more of other computing devices, such as computing devices of or associated with imaging system(s) 108 and/or controller 106, may include the same or similar application to perform at least a portion (or all) of the operations of FIG. 4 (discussed below). In some examples, one application operating on a single computing device of environment 100 may be configured to perform each of the steps. In other examples, multiple applications running on a same or across different computing devices of environment 100 may perform different operations. As one non-limiting example, one application may be used to receive velocity and distance measurements of a distal tip of medical device 102 from distal tip sensor(s) 112, while a second application may be used to receive velocity and distance measurements of a proximal portion of the medical device 102 via proximal sensor(s) 110. Yet another may application may be used to generate and display an alert or other information on the GUI.

The process 400 occurs during an endoscopic procedure, and may begin with the insertion of the insertion portion 204 of medical device 102 into a patient. Some or all measurements made in the process 400 may be timestamped, with a timestamp of t=0 starting at the time of the start of the procedure. In some examples, process 400 may be performed by one or a combination of components of environment 100, such as controller 106, via the one or more applications of controller 106 or the components of controller 106. For example, steps of process 400 may be performed by position sensing module 116 or computing device 118.

At step 402, controller 106 (e.g., position sensing module 116) may receive velocity and/or distance measurements from the proximal sensor(s) 110. The velocity measurement from the proximal sensor(s) 110 represents, in at least some examples, the velocity of shaft 208 of insertion portion 204 at a proximal position where proximal sensor(s) 110 are situated. The distance measurement may represent the length of insertion portion 204 that has passed by the position of proximal sensor(s) 110 (a distance between distal tip 210 and proximal sensor 110) in a time t from the beginning of the procedure.

Proximal sensor(s) 110 may measure any or all of acceleration, velocity, or distance traveled of/by shaft 208 directly (e.g., without being inferred or calculated from other measurements). Alternatively, proximal sensors 110 may measure one of acceleration, velocity, or distance traveled of/by shaft 208 directly and include onboard programming or applications to calculate (determine indirectly) the remaining terms. For example, proximal sensor(s) 110 may measure acceleration directly based on the timestamped measurements of passing stripes, and may calculate the velocity of the proximal portion of the insertion tube by calculating the first integral of the measured acceleration with respect to a measured time, and calculating a distance traveled of the insertion tube by calculating a double integral of the measured acceleration with respect to a measured time. Proximal sensor(s) 110 may also directly measure distance traveled of the insertion tube and calculate a velocity of the insertion tube at a proximal portion by calculating a first derivative of the distance traveled with respect to time and acceleration by calculating a second derivative of the distance traveled over time. Proximal sensor(s) 110 may transmit one or more of acceleration, velocity, and distance measurements to controller 106 (e.g. position sensing module 116) to calculate the remaining terms. The measurements from proximal sensor(s) 110 may be transmitted to controller 106 (and/or vice versa) via a wired connection, Bluetooth, Wifi, or any other suitable modality.

At step 404, controller 106 (e.g., position sensing module 116) may receive velocity, distance, and/or acceleration measurements from the distal tip sensor(s) 112. The velocity, distance and acceleration measurements from distal tip sensor(s) 112 represent the velocity of distal tip 210 of insertion portion 204 within a lumen of patient P.

Distal tip sensor(s) 112 may measure any or all of acceleration, velocity, or distance traveled directly. Proximal sensor(s) 110 may also measure one of acceleration, velocity, or distance directly and components of controller 106, such as position sensing module 116 may calculate any other desired variables. For example, proximal sensor(s) 110 may measure acceleration directly based on the signals generated by on-board gyroscopes, accelerometers, and/or magnetometers, such as those of IMUs. The controller 106 (e.g., position sensing module 116) may calculate the velocity of distal tip 210 of insertion portion 204 by calculating the first integral of the measured acceleration with respect to a measured time. Controller 106 (e.g., position sensing module 116) may calculate a distance traveled of the insertion tube by calculating a double integral of the measured acceleration with respect to a measured time. Distal tip sensor(s) 112 may additionally or alternatively directly measure distance traveled of the insertion tube and controller 106 (e.g., position sensing module 116) may calculate a velocity of the insertion tube at by calculating a first derivative of the distance traveled with respect to time. Controller 106 (e.g., position sensing module 116) may further calculate an acceleration of the insertion tube by calculating a second derivative of the distance traveled with respect to time.

Steps 402 and 404 may occur simultaneously or in reverse order.

After the measurements are received in steps 402 and 404, the controller 106 (e.g., position sensing module 116) may use an application or programming to compare the measurements received from the proximal sensor(s) 110 (step 402) and distal tip sensor(s) 112 (step 404). At step 406, the velocity measured at a proximal position by proximal sensor(s) 110 may be compared with the velocity measured at the distal tip by distal tip sensor(s) 112. The velocity measured at a proximal position and the velocity measured at the distal tip may both be continuously determined. For example, steps 402 and 406 may be performed continuously. The difference between the velocity measured at the proximal position and the velocity measured at the distal tip may be continuously determined, or may be calculated at regular intervals, such as every 0.1 seconds, 0.5 seconds, etc.

A difference between the velocity of the proximal portion and the velocity of the distal tip may be indicative of looping or bowing of medical device 102. For example, if the velocity measured at the proximal portion is positive, for example 1 cm/sec, this is indicative of insertion of medical device 102 further into the lumen of patient P. If the velocity measured at the distal tip at the same time is less than 1 cm/sec, this indicates that the distal tip is not progressing through the lumen of the patient P at the same rate that medical device 102 is being inserted into patient P. A distal tip velocity of, for example, 0.5 cm/sec, may indicate that the medical device is experiencing moderate levels of looping or bowing that may or may not resolve itself. A velocity of 0 cm/sec indicates that the distal tip is not advancing at all, such that a user may suspect significant looping or bowing. Further, a negative velocity indicates that medical device 102 is likely distending the lumen of patient P in a manner that is almost assuredly a looping or bowing event.

Although determining a difference between the velocity of the proximal portion and the velocity of the distal tip is discussed above, it will be appreciated that other types of calculation (e.g., ratios, percentages, etc.) may instead be performed in step 406.

At step 408, the distance measured at a proximal position by proximal sensor(s) 110 may be compared with the distance measured at the distal tip by distal tip sensor(s) 112. Similarly to the velocity, the distance measured at a proximal position and the distance measured at the distal tip may both be continuously determined. Likewise, the difference between the distance measured at the proximal position and the distance measured at the distal tip may be continuously determined, or may be calculated at regular intervals, such as every 0.1 seconds, 0.5 seconds, etc.

A difference between the distance of the proximal portion and the distance of the distal tip may be indicative of looping or bowing of medical device 102. For example, if the distance measured at the proximal portion is positive, for example 5 cm, this is indicative of insertion of medical device 102 further into the lumen of patient P. If the distance measured at the distal tip at the same time is less than 5 cm, this indicates that the distal tip is not progressing through the lumen of the patient P the same distance that medical device 102 is being inserted into the patient P. A distal tip distance of, for example, 1 cm, may indicate that the medical device is experiencing moderate levels of looping or bowing that may or may not resolve itself. A distal tip distance of 0 cm indicates that the distal tip is not advancing at all, such that a user may suspect significant looping or bowing. Further, a negative distance traveled at the distal tip indicates that medical device 102 is likely distending the lumen of patient P in a manner that is almost assuredly a looping or bowing event.

Although determining a difference between the distance of the proximal portion and the distance of the distal tip is discussed above, it will be appreciated that other types of calculation (e.g., ratios, percentages, etc.) may instead be performed in step 406.

In some embodiments, only one or the other of the differences (difference in velocity or difference in distance) may be determined. For example, in some embodiments only step 406 is performed before proceeding to step 410. In other embodiments, only step 408 is performed before proceeding to step 410. In some examples, step 406 and 408 may both be performed (e.g., simultaneously or serially). In some examples, one of steps 406 or 408 may be performed before the other of steps 406 or 408. The other of steps 406 or 408 may be performed only if a result of the first-performed step 406 or 408 exceeds a predetermined threshold.

At step 410, it is determined whether the differences measured in one or both of steps 406 and 408 exceed a predetermined threshold. As discussed above, a difference between the velocity measured at the proximal position and the velocity measured at the distal tip may be indicative of a bowing or looping event. Similarly, a difference between the distance measured at the proximal position and the distance measured at the distal tip may be indicative of a bowing or looping event.

A predetermined threshold for the difference (or other calculation) between the velocity of the proximal portion and the velocity of the distal tip may be set by a user or based on a rules-based or machine learning model. Similarly, a predetermined threshold for the difference between the distance of the proximal portion and the distance of the distal tip may be set by a user or based on a rules-based or machine learning model. One or both of the thresholds may be set based on a sensitivity preference to identifying potential bowing or looping incidents. Smaller thresholds are more sensitive but may yield more false positives, while larger thresholds are less likely to yield false positives but may be less likely to detect bowing or looping incidents at an early stage. For example, a threshold may be set to trigger an alert if the velocity difference between a distal tip and a proximal portion of the medical device is larger than approximately 1.0 cm/s, or if a difference in distance traveled of the distal tip and the proximal portion is larger than approximately 2.0 cm.

The predetermined thresholds may also be set to be large enough to account for noise in the measurements and “drift,” which is a cumulative error rate in determining a measurement based on compounding rounding errors in calculations, particularly in the threshold for the difference between the distance measured at the proximal portion and the distance measured at the distal tip. In some examples, distal tip sensor(s) 112 include an IMU, which measures acceleration at the distal tip. In these examples, the velocity and distance measurements at the distal tip are determined by integrating and double integrating, respectively, the acceleration measurements at the distal tip. In each integration step, some rounding error may be introduced. Because distance is a double integration of acceleration, over time this rounding error may be compounded such that the distance measurement “drifts” away from a true distance traveled based on the compounding rounding errors introduced in the double integration calculation.

In addition to defining a threshold difference, another technique to account for factors such as noise or drift may be to use an algorithm that determines a difference in a specified time window. The specified time window may, for example, be the most recent 1.0, 2.0, 3.0, etc. seconds of the procedure. A sudden measured difference in distance traveled between the distal end and a proximal portion of an insertion portion of an endoscope is highly correlated with a possible bowing or looping event. As such, in addition to considering a difference between proximal and distal velocities and/or proximal and distal distances traveled, step 408 may consider a time period over which such differences ocurred. For example, a bowing or looping incident may be indicated if the difference between the distance traveled at the distal end and the distance traveled at a proximal position is greater than 1 cm, and the difference persists for a time period of 3 seconds.

In some examples, as discussed above, one of steps 406 or 408 may be performed, and then step 410 may be performed. If 410 produces a result exceeding the threshold, then the other of steps 406 or 408 may be performed, and step 410 may be repeated for the other type of measurement.

At step 412, a bowing or looping alert may be initiated by controller 106 (e.g., position sensing module 116) based on the results of step 408. The alert may include one or more of a visual, auditory, or tactile alert. Accompanying or in addition to the alert may be an automatic provision of a treatment, as depicted in step 414, which may include the use of a robotic scope to adjust medical device 102 to prevent the looping or bowing. In some examples, a user and/or medical professional may be alerted on a GUI 500 of a display device, as depicted in step 416. Although optional step 414 is depicted before step 416, it will be appreciated that step 416 may be performed before step 414.

FIG. 5 depicts an exemplary GUI 500 generated and displayed during a medical procedure, such as a gastrointestinal endoscopy procedure. The generated GUI may be displayed on display 120 of FIG. 1. The GUI 500 may display a current image 502 of the lumen of a patient P, for example, captured by imaging device 212 of medical device 102 during the procedure. Over the normal course of the procedure, image 502 may take up an entire available display space or a majority of available display space on the GUI 500. However, when a bowing or looping alert is generated, the GUI 500 may reduce the size of the image 502 of the lumen to provide space for alert images 504-510.

A first alert image 504 may include text indicating that bowing or looping is detected. For example, the text may include “ALERT! LOOPING OR BOWING DETECTED” as shown in FIG. 5. First alert image 504 may be displayed in a color meant to catch the attention of a user, such as red or yellow, and may include image objects such as an exclamation point or other indicators of urgency. The first alert image 504 may be configured to flash in the display and may be accompanied by an audible alert. A second alert image 506 and a third alert image 508 may depict graphs of the velocity vs. time and the distance measured vs. time, respectively, of the proximal and distal portions of the insertion portion 204 as measured by the proximal sensor(s) 110 and the sensor(s) 133. These alert images 506 and 508 may be displayed throughout the procedure, or displayed as a result of the alert being triggered in step 412. Displaying the images 506 and 508 may be a user option. The options may include displaying the images 506 and 508 throughout the procedure or displaying the images 506 and 508 upon the trigger of a looping or bowing alert. The images 506 and 508 may provide a user with information that may be used to investigate whether the alert is a genuine concern or a potential false alarm.

Examples of images 506 and 508 may include a graph of the velocity of the proximal portion and the velocity of the distal tip with respect to time and/or a graph of the distance of proximal portion and the distance of the distal tip with respect to time. In graph 506, the velocities are on the y-axis and time on the x-axis, with a call out image indicating a portion in the graph that highlights where the alert occurred. A user may view the image and see the information regarding the difference between the proximal portion velocity and the distal tip velocity to help in their investigation as to the nature of the alert. In graph 508, the distances are on the y-axis and time on the x-axis, with a call out image indicating a portion in the graph that highlights where the alert occurred. A user may view the image and see the information regarding the difference between the proximal portion distance and the distal tip distance to help in their investigation as to the nature of the alert. A call out portion may also indicate where drift is identified. Alert image 510 may provide an additional visual cue as to what the alert represents, with a graphical depiction of looping or bowing shown.

Although not shown in FIG. 5, various other visual schemes may be employed within GUIs generated and displayed during a medical procedure. The GUI 500 described above is provided merely as an example, and may include additional, fewer, different, or differently arranged information and/or features than depicted in FIG. 5.

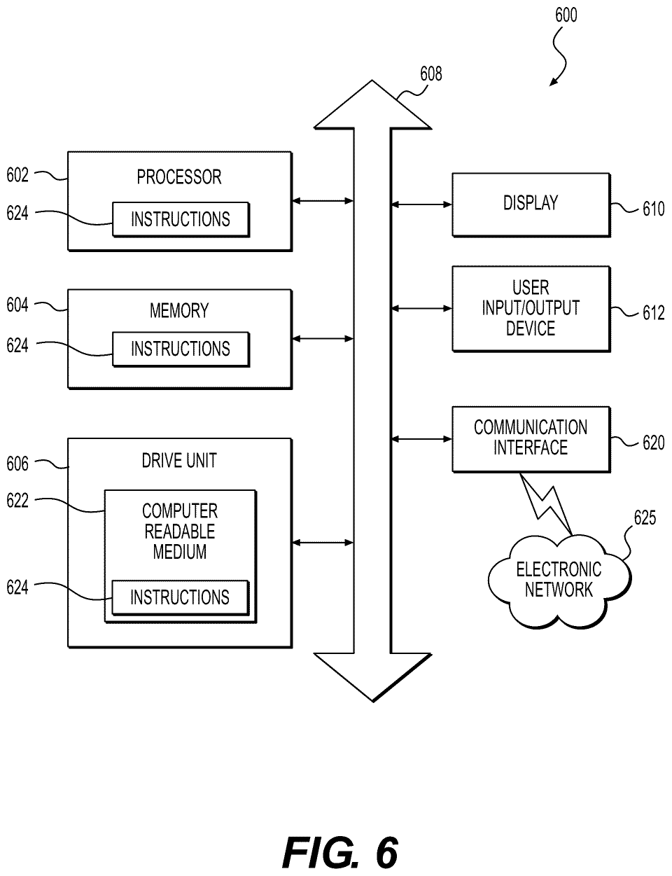

FIG. 6 depicts an example of a computer 600. FIG. 6 is a simplified functional block diagram of computer 600 that may be configured as a device for executing processes, steps, or operations depicted in, or described with respect to, FIGS. 1-5 and, according to exemplary embodiments of the present disclosure. For example, computer 600 may be configured as one of a computing device of or associated with controller 106 and/or another device or component according to exemplary embodiments of this disclosure. In various embodiments, any of the systems herein may be or include computer 600 including, e.g., a data communication interface 620 for packet data communication. Computer 600 may communicate with one or more other computers, for example, using an electronic network 625 (e.g., via data communication interface 620). Electronic network 625 may include a wired or wireless network similar to network 114 depicted in FIG. 1.

Computer 600 also may include a central processing unit (“CPU”), in the form of one or more processors 602, for executing program instructions 624. Computer 600 may include an internal communication bus 608. The computer may also include a drive unit 606 (such as read-only memory (ROM), hard disk drive (HDD), solid-state disk drive (SDD), etc.) that may store data on a computer readable medium 622 (e.g., a non-transitory computer readable medium), although computer 600 may receive programming and data via network communications. Computer 600 may also have a memory 604 (such as random-access memory (RAM)) storing instructions 624 for executing techniques presented herein. It is noted, however, that in some aspects, instructions 624 may be stored temporarily or permanently within other modules of computer 600 (e.g., processor 602 and/or computer readable medium 622). Computer 600 also may include user input and output devices 612 and/or a display 610 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A system for detecting bowing or looping events during a medical procedure, the system comprising:
a medical device including:
an insertion portion having a proximal portion and a distal end; and
a distal sensor disposed at the distal end of the insertion portion, wherein the distal sensor is configured to measure an acceleration or a velocity of the distal end of the insertion portion or a distance traveled by the distal end of the insertion portion; and
a proximal sensor configured to measure an acceleration or a velocity of the proximal portion of the insertion portion or a distance traveled by the proximal portion of the insertion portion.

2. The system of claim 1, wherein the distal sensor is an inertial measurement unit (IMU).

3. The system of claim 1, wherein the proximal sensor includes a fiber optic sensor or a camera.

4. The system of claim 3, wherein the insertion portion includes a plurality of stripes, wherein a first set of the plurality of stripes has a first property, wherein a second set of the plurality of stripes has a second property, and wherein the second property differs from the first property.

5. The system of claim 4, wherein the proximal sensor is configured to sense a passing of the plurality of stripes to determine the distance traveled by the proximal portion of the insertion portion.

6. The system of claim 4, wherein the proximal sensor is configured to sense a passing of the plurality of stripes to determine the velocity of the distal end.

7. The system of claim 1, wherein the proximal sensor is attached to a patient's skin.

8. The system of claim 1 further comprising: a controller configured to compare one or more of the acceleration, the velocity, or the distance measured by the distal sensor to the acceleration, the velocity, or the distance measured by the proximal sensor.

9. The system of claim 8, wherein the comparison includes determining one or more of (a) an acceleration difference between the acceleration measured by the proximal sensor and the acceleration measured by the distal sensor, (b) a velocity difference between the velocity measured by the proximal sensor the velocity measured by the distal sensor, or (c) a distance difference between the distance measured by the proximal sensor and the distance measured by the distal sensor.

10. The system of claim 9, wherein the controller is further configured to compare one or more of the acceleration difference, the velocity difference, or the distance difference to a respective threshold.

11. The system of claim 10, wherein the controller is configured to determine that looping or bowing of insertion portion is occurring upon determining that one of more of the acceleration difference, the velocity difference, or the distance difference is greater than the respective threshold.

12. The system of claim 10, wherein the controller is configured to generate an alert upon determining that one of more of the acceleration difference, the velocity difference, or the distance difference is greater than the respective threshold, wherein the alert is auditory, visual, or tactile.

13. The system of claim 10, wherein the controller provides instructions to automatically adjust a scope upon determining that one of more of the acceleration difference, the velocity difference, or the distance difference is greater than a respective threshold.

14. The system of claim 9, wherein the controller is configured to determine that looping or bowing of insertion portion is occurring upon determining that the difference between the proximal distance the distal distance is greater than a predetermined threshold for a predetermined interval of time.

15. The system of claim 1, wherein the insertion portion is configured to be inserted into a patient's body and wherein the proximal sensor is configured to remain outside of the patient's body.

16. A system for detecting bowing or looping events during a medical procedure, the system comprising:
a medical device including an insertion portion having a proximal portion and a distal portion, wherein the distal portion includes a first sensor configured to measure a first parameter of the distal portion;
a second sensor configured to measure a second parameter of the proximal portion;
a controller configured to compare the second parameter and the first parameter in order to determine whether looping or bowing of the insertion portion has occurred or is occurring.

17. The system of claim 16, wherein the first parameter is distance, velocity or acceleration and the second parameter is distance, velocity, or acceleration.

18. A medical method comprising:
receiving a proximal measurement from a proximal sensor, wherein the proximal measurement indicates a velocity or acceleration of a proximal portion of an insertion portion of a medical device or a distance traveled by the proximal portion of the insertion portion of the medical device;
receiving a distal measurement from a distal sensor, wherein the distal measurement indicates a velocity or acceleration of a distal portion of an insertion portion of a medical device or a distance traveled by the distal portion of the insertion portion of the medical device;
comparing the proximal measurement to the distal measurement;
based on the comparison between the proximal measurement and the distal measurement, determine that a bowing event or a looping event has occurred or is occurring; and
providing an alert that the bowing or looping event has occurred or is occurring.

19. The method of claim 18, wherein the comparing includes determining a difference between the proximal measurement and the distal measurement.

20. The method of claim 19, further comprising: comparing the difference to a predetermined threshold.
